(19) United States
(12) Patent Application Publication
(10) Pub. No.: US 2010/0331599 A1
(43) Pub. Date: Dec. 30, 2010

(54) ALKYLATION CATALYZED BY BINARY MIXTURES OF ACID AND IONIC LIQUID

(76) Inventors: Bala Subramaniam, Lawrence, KS (US); Aaron Scurto, Oskaloosa, KS (US); Shengwei Tang, Chengdu (CN)

Correspondence Address: Workman Nydegger;1000 Eagle Gate Tower, 60 East South Temple, Salt Lake City, UT, 84111, US

(21) Appl. No.: 12/797,450

(22) Filed: Jun. 9, 2010

Related U.S. Application Data
(60) Provisional application No. 61/185,488, filed on Jun. 9, 2009.

(51) Int. Cl. C07C 2/58; B01J 37/30

(52) U.S. Cl. 585/730; 502/11

(57) ABSTRACT

An alkylation catalyst can include: a Brønsted acid ionic liquid; and a strong Brønsted acid that is not considered an ionic liquid. The Brønsted acid ionic liquid can be selected from the group consisting of [BMIm]HSO₄, [MBSIm]HSO₄, [MBSIm]OTf, [MPSIm]OTf, and [OMIm]HSO₄ or the like. In one aspect, the strong Brønsted acid is selected from the group consisting of sulfuric acid, hydrochloric acid (HCl), hydrobromic acid (HBr), HF, hydrogen iodide (HI), phosphoric acid, trifluoromethanesulfonic (triflic) acid. In one aspect, the strong Brønsted acid is present at more than about 50 wt % of the composition; however, the Brønsted acid can vary from about 10 wt % to about 99 wt %, more preferably about 20 wt % to about 90 wt %, and most preferably about 40 wt % to about 80 wt %.

### CROSS-REFERENCE

This patent application claims benefit of U.S. provisional patent application 61/185,488, filed Jun. 9, 2009, which provisional application is incorporated herein by specific reference in its entirety.

### BACKGROUND

Alkylation of isobutane with various olefins is a major process in the petroleum industry for the production of gasoline. The alkylation of C₃ to C₅ olefins with isobutane produces a mixture of highly branched alkanes, mainly isooctanes. This alkylate offers a high octane number, a low Reid vapor pressure (RVP) and low octane sensitivity [e.g., difference between research octane number (RON) and motor octane number (MON)]. It contains no aromatics and nearly no sulfur. This alkylate is usually blended with various cuts of refined crude oil.

Current commercial alkylation processes are catalyzed by concentrated sulfuric acid (H₂SO₄) or hydrofluoric acid (HF). The sulfuric acid process produces large amounts of spent acid and acid soluble oils. The spent acid is costly to regenerate. Anhydrous HF is highly toxic and its leakage results in dangerously stable aerosols at the ground level. In addition, equipment corrosion, transport, handling of hazards and environmental liability associated with the disposal of spent acid are disadvantages for both processes. Hence, efforts continue to develop efficient alternative processes that reduce the amount of acid use and are relatively safer.

### SUMMARY

In one embodiment, the present invention can include an alkylation catalyst composition comprising: an acid ionic liquid, such as a protic ionic liquid or a Brønsted acid ionic liquid; and a strong Brønsted acid that is not considered an ionic liquid. In one aspect, the acid ionic liquid can include a [Cation][Acidic Anion] such as [Cation][HSO₄], [Cation][OTf], [Cation][Tf₂N], [Cation][H₂PO₄], [Cation][HPO₄]⁻², [Cation][HCO₃], [Cation][Acidic Inorganic Anion], [Cation][H₂VO₄] or the like, with the [Cation] being an imidazolium, such as a substituted imidazolium. An example of substituted imidazoliums of the acid ionic liquid can include substitutions with pyridinium, ammonium, phosphonium, and the like.

In one aspect, the protic ionic liquid can be selected from the group consisting of (a) primary, secondary, or tertiary ammonium cations; (b) 1-alkylimidazolium cations; (c) 1-alkyl-2-alkylimidazolium cations; (d) caprolactam; and (e) 1,1,3,3-tetramethylguanidinine. Representative anions, including: (a) carboxylates; (b) trifluoroacetate (TFA); (c) bis(perfluoroethylsulfonyl)imide (BETI); (d) bis(trifluoromethanesulfonyl)imide (TFSI); (e) nitrate; and (f) hydrogen sulfate.

In one aspect, the Brønsted acid ionic liquid is selected from the group consisting of [BMIm]HSO₄ (1-butyl-3-methylimidazolium hydrogen sulphate), [MBSIm]HSO₄ (1-methyl-3-(butyl-4-sulfonate)imidazolium hydrogen sulphate), [MBSIm]OTf (1-butyl-3-(butyl-3-sulfonyl)imidazolium trifluoromethanesulfonate), [MPSIm]OTf (1-butyl-3-(butyl-3-sulfonyl)imidazolium trifluoromethanesulfonate), and [OMIm]HSO₄ (1-octyl-3-methylimidazolium hydrogen sulphate). The [HSO₄] may be replaced with [H₂PO₄], [HPO₄]⁻², bicarbonates [HCO₃], triflate anion [CF₃SO₃], dichromic [HCr₂O₇], chromic [HCrO₄], silicic [HSiO₃], benzenesulfonate anion [C₆H₅SO₃], boric [H₂BO₃] or [HBO₃], acidic inorganic anions, vanadate [H₂VO₄] from vanadic acid, or the like. Also, the imidazolium of the Brønsted acid ionic liquid can be a substituted imidazolium, such as substitutions with pyridinium, ammonium, phosphonium, and the like.

In one aspect, the strong Brønsted acid is a superacid. The superacid can be selected from the group consisting of sulfuric acid, hydrochloric acid (HCl), hydrobromic acid (HBr), HF, hydrogen iodide (HI), phosphoric acid, trifluoromethanesulfonic (triflic) acid, fluoro-sulfonic acid (HSFO₃), 1,1,1-trifluoro-N-(trifluoromethylsulfonyl)methanesulfonamide (H(Tf2N)), or the like. In one aspect, the superacid can have a Hammett number less than about negative ten (e.g., H₀<−10).

In one aspect, the strong Brønsted acid is present at more than about 50 wt % of the composition; however, the Brønsted acid can vary from about 10 wt % to about 99 wt %, more preferably about 20 wt % to about 90 wt %, and most preferably about 40 wt % to about 80 wt %.

In one embodiment, the alkylation catalyst composition is substantially devoid of a weak and/or medium Brønsted acid; and/or substantially devoid of a neutral ionic liquid; and/or substantially devoid of a Lewis acid ionic liquid, as defined. These substances can be excluded in alkylation reactions of isoparaffins. However, some neutral ionic liquid become acidic in the mixture with the acidic ionic liquid or strong acid, and such neutral ionic liquids can be included in alkylation reactions of isoparaffins. Also, any of these substances can be included in other types of reactions (e.g., other than alkylation of isoparaffin) using the acidic ionic liquid and Brønsted acid, such as Friedel-Krafts alkylation, acylation chemistries, or other acid-catalyzed reaction.

In one embodiment, the alkylation catalyst composition can be included in an alkylation reaction mixture along with a paraffin, such as an isoparaffin, and an olefin. In one aspect, the olefin can be selected from the group consisting of propylene, pentene, isobutylene, 1-butene, trans-2-butene, cis-2-butene, and derivatives thereof. The isoparaffin can be selected from the group consisting of isobutane and isopentane. In one aspect, the isoparaffin:olefin (“I:O”) ratio can be 1:1, 2:1, 3:1, 4:1, 5:1, 6:1, 7:1, 8:1, 9:1, 10:1, 11:1, 12:1, 13:1, 14:1, 15:1, vice versa, or any range between a combination of the foregoing. The paraffin:olefin (“P:O”) ratios can be the same as the I:O ratios.

In one embodiment, the alkylation reaction mixture can be used in a process for alkylating a paraffin, such as an isoparaffin. The alkylation reaction can include: providing a reaction mixture as described herein; reacting the isoparaffin and an olefin in the presence of the alkylation catalyst; forming an alkylated isoparaffin product; and recovering an alkylated isoparaffin product from the reaction mixture.

In one aspect, the reacting step can occur at a temperature of about −10° C. to about 50° C., but can vary from about −5° C. to about 40° C., more preferably about 0° C. to about 20° C., and most preferably about 3° C. to about 10° C.

In one aspect, the reacting step can occur at a pressure of about 1 bar to about 60 bar, but can vary from about 5 bar to about 40 bar, more preferably about 10 bar to about 30 bar, and most preferably about 15 bar to about 25 bar.

The process can have C₈ selectivity which is greater than 50%, but can vary from about 50% to about 80%, more preferably about 60% to about 90%, and most preferably about 70% to about 95%. C₈ selectivity is defined as the weight of C₈ products divided by the weight of all products. The alkylation reaction can be conducted in a batch, semi-continuous, or continuous manner.

In one embodiment, the alkylation reaction can include a step of recovering the alkylation catalyst from the reaction mixture. Such recovery can be almost complete but can accommodate some contaminants from the reaction yet sufficiently pure to perform the subsequent alkylation. For example, the extent of catalyst recovery can be between 90 and 100%, with the remaining being made up by fresh supply.

In one embodiment, the alkylation reaction can include a step of recycling the recovered alkylation catalyst into a second reaction mixture with fresh isoparaffin and olefin.

In one embodiment, the alkylation reaction can include a step of recycling the recovered alkylation catalyst at least 5 times. The reactions with recycled alkylation catalyst can have a conversion of at least 50% in each cycle but can vary from about 50% to about 100%, more preferably about 70% to about 95%, and most preferably about 90% to about 99%.

In one embodiment, the process has trimethylalkyl:dimethylalkyl ratio equal to or greater than about 4:1, such as trimethylpentane:dimethylhexane (“TMP:DMH”) ratio of greater than 4:1 but can vary from about 4:1 to about 10:1, more preferably about 5:1 to about 7:1, and most preferably about 6:1 to about 8:1.

The foregoing summary is illustrative only and is not intended to be in any way limiting. In addition to the illustrative aspects, embodiments, and features described above, further aspects, embodiments, and features will become apparent by reference to the drawings and the following detailed description. In many instances it is conceived that paraffin can be used in place of isoparaffin; however, it is expected that isoparaffins may be optimal and have surprising and unexpected improvements.

### BRIEF DESCRIPTION OF THE FIGURES

FIG. 1 includes structures of some examples of cations and anions of the ionic liquids.

FIG. 2 includes a schematic of an embodiment of a reactor system 100 for alkylation. As shown, the reactor system 100 includes a feed of reactant 102, a pump 104, a reactor vessel 106, a temperature control system 108, a pressure control system 110, a stirring system 112, and outlet 114, a purification system 116, a gas vent 118, and recovery unit 120 that produces pure product 122 and regenerated catalyst 124.

FIGS. 3A-3B include graphs that show the effects of catalyst reuse on butene conversion: C₈ selectivity (FIG. 3A) and TMP/DMH ratio (FIG. 3B). The catalyst included: 33.2 g of [OMIm]HSO₄ (16 wt %)+H₂SO₄ (84 wt %) or 26.6 g of H₂SO₄. The reaction parameters included: Initial Temperature=8° C.; Pressure ˜2 bar; Batch reaction time=10 min; Volume of hydrocarbon fed=20 mL with a molar I/O in feed=7.5. The legend for FIGS. 3A-3B includes: ♦-conversion, mixture; □-conversion, H₂SO₄; ▴-C₈ selectivity, mixture; Δ-C₈ selectivity, H₂SO₄; -TMP/DMH, Mixture; ∘-TMP/DMH, H₂SO₄.

FIGS. 4A-4B include graphs that show a comparison of the reuse of catalysts based on trifluoromethanesulfonic acid C₈ selectivity (FIG. 4A) and TMP/DMH ratio (FIG. 4B). The catalyst included: Either 26 g of a mixture of [OMIm]HSO₄ (22.6 wt %)+77.4 wt % HCF₃SO₃ or 30 g of mixture of 93.2 wt % HCF₃SO₃ and 6.8 wt % H₂O or 33.9 g of neat HCF₃SO₃. The reaction parameters included: Initial temperature: 8° C.; Pressure: ˜2 bar; Batch reaction time=10 min; Volume of hydrocarbon fed=20 mL with a molar I/O in feed=7.5. The legend includes: ♦-conversion, Mixture of [OMIm]HSO₄+HCF₃SO₃; □-conversion, mixture of H₂O+HCF₃SO₃; *-conversion, HCF₃SO₃; ▴-C₈ selectivity, mixture of [OMIm]HSO₄+HCF₃SO₃; Δ-C₈ selectivity, mixture of H₂O+HCF₃SO₃; ⋄-C₈ selectivity, HCF₃SO₃; -TMP/DMH, Mixture of [OMIm]HSO₄+HCF₃SO₃; x-TMP/DMH, Mixture of H₂O+HCF₃SO₃; ∘-TMP/DMH, HCF₃SO₃.

FIGS. 5A-5B include graphs of a comparison of performance of [OMIm]HSO₄+HCF₃SO₃ mixtures at different I/O ratios: C₈ selectivity (FIG. 5A) and TMP/DMH ratio (FIG. 5B). The catalyst were used at I/O=10 and include: 25.1 g of [OMIm]HSO₄ (23.7 wt %+HCF₃SO₃ (76.3 wt %). The catalyst that were used at I/O=7.5 include: 26.1 g of [OMIm]HSO₄ (22.6 wt %+HCF₃SO₃ (77.4 wt %). The reaction parameters included: Temperature=8° C.; Pressure=˜2 bar; Batch reaction time=10 min; Volume of hydrocarbon feed=20 mL.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Broadly, the catalyst composition can be used in an alkylation processes to bring paraffins, isoparaffins, or aromatics and light olefins into intimate contact with an acid catalyst to produce an alkylation product. The alkylation process can involve the reaction of light olefins (such as propylene and butylene) with isoparaffins (such as isobutane and isopentane) in the presence of an acid catalyst (such as H₂SO₄ and HF) to form highly branched, isoparaffinic products known as alkylates. Alkylation can include the reaction of a paraffin, usually isoparaffins, with an olefin in the presence of a strong acid which produces paraffins with higher octane number than the starting materials. The produced paraffins also boil in range of gasolines.

Generally, the present invention includes the alkylation of paraffin hydrocarbons with olefin hydrocarbons with a catalyst composition having an ionic liquid and a strong acid. More particularly, the present invention includes alkylation of isoparaffins with olefins with acidic ionic liquids and strong acids, more particularly with acidic ionic liquids and strong Brønsted acids, even more particularly with protic ionic liquids or Brønsted acid ionic liquids and strong Brønsted acids.

An example is with 1-butene/isobutane alkylation to yield C₈-alkylates (“C₈” being 8 carbon) using catalyst compositions that are binary mixtures of certain acidic imidazolium ionic liquids (ILs) and strong acids such as sulfuric or trifluoromethanesulfonic (triflic) acid. The process can obtain equivalent or better conversion (>95%), C₈ alkylates selectivity (>70%), and trimethylpentane/dimethlyhexane selectivity (TMP/DMH>7), compared to acid or ionic liquid alone. These improvements can be achieved with the ionic liquid/acid mixtures over the conversion or selectivity of the pure acids. The present invention provides the unexpected synergistic effects of binary ionic liquid and strong acid mixtures for 1-butene/isobutane alkylation with improved conversion, selectivity, and recyclability.

In addition, the catalyst composition can be applicable to Friedel-Crafts alkylation, acylation chemistries, or other acid-catalyzed reaction.

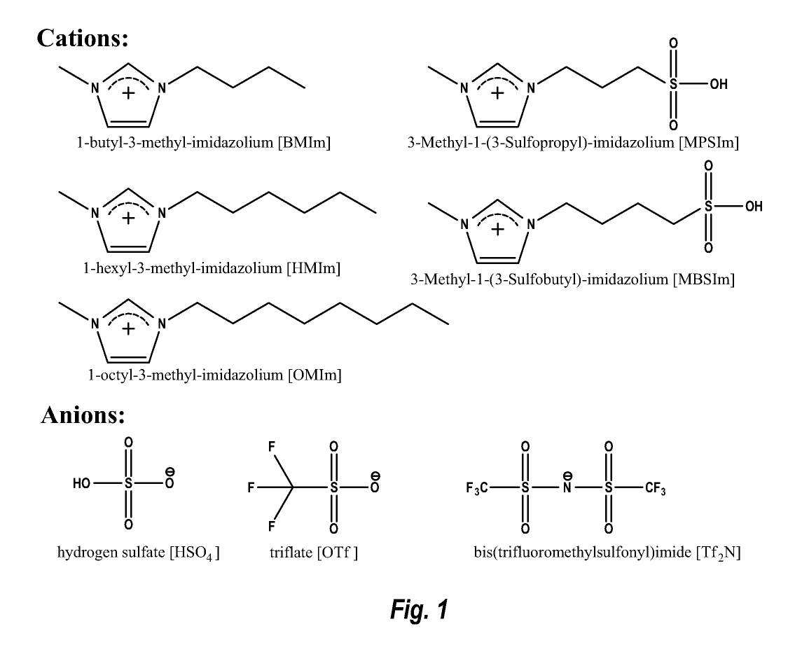

While any ionic liquid is envisioned, especially imidazolium ionic liquids, protic protic ionic liquids, and Brønsted acid ionic liquids, to be capable of being used in the process, 5 types of substituted 3-methyl-imidazolium ionic liquids were found to be particularly useful. These ionic liquids were [BMIm]HSO₄ (1-butyl-3-methylimidazolium hydrogen sulphate), [MBSIm]HSO₄ (1-methyl-3-(butyl-4-sulfonate)imidazolium hydrogen sulphate), [MBSIm]OTf (1-butyl-3-(butyl-3-sulfonyl)imidazolium trifluoromethanesulfonate), [MPSIm]OTf (1-butyl-3-(butyl-3-sulfonyl)imidazolium trifluoromethanesulfonate), and [OMIm]HSO₄ (1-octyl-3-methylimidazolium hydrogen sulphate). These ionic liquids can be prepared from the cations and anions of FIG. 1. Also, the envisioned ionic liquids can have acidity imparted via a cation with sulfonic acid groups or an anion such as hydrogen sulfate, or even both.

The acidic ionic liquid can be a protic ionic liquid, where the acidic proton comes from the cation. The cation is matched with an anion, such as those described herein. Examples include [Cation][Acidic Anion], [Cation][HSO₄], [Cation][OTf], [Cation][Tf₂N], [Cation][H₂PO₄], [Cation][HPO₄]⁻², [Cation][HCO₃], [Cation][Acidic Inorganic Anion], [Cation][H₂VO₄] or the like. The cation can be an imidazolium, such as a substituted imidazolium. Also, the cation can be shown by Structure 1, Structure 2, Structure 3, or other similar cationic structure. In Structures 2-3, the R groups can be independently selected from pyridiniums, ammoniums, phosphoniums, derivatives thereof, and the like. Also, another acid group (e.g., sulfonic acid, etc.) can be added to the R group on the cation of Structures 2 and 3. If the proton is removed, a neutral IL-precursor is formed. For instance, Structure 1 upon dissociation becomes an acidic proton and neutral butyl-imidazole. These compounds can be effective as long as they are not exposed to basic conditions. This ability to switch from neutral precursor to cation can yield significant processing advantages especially when the acid catalyst needs to be regenerated. For instance the ionic form has no vapor pressure and thus is not distillable, while butyl-imidazole is volatile and can be distilled off.

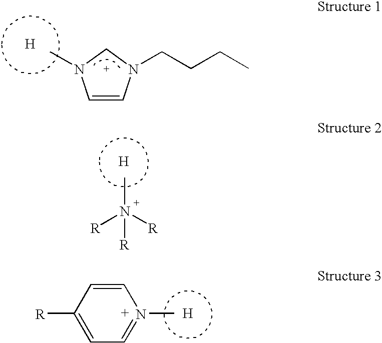

While it is conceived that the strong acid can be any strong acid, it has been found that sulfuric acid, HCl, HBr, HF, HI, phosphoric acid, trifluoromethanesulfonic (triflic) acid, fluoro-sulfonic acid (HSFO₃), 1,1,1-trifluoro-N-(trifluoromethylsulfonyl)methanesulfonamide (H(Tf2N)), or the like. In one aspect, the superacid can have a Hammett number less than about negative ten (e.g., H₀<−10). More preferably, the strong acid is sulfuric acid and/or triflic acid, which provide surprising and unexpected improvements.

Preparation of the catalyst composition can be performed by modulating the ratio of the ionic liquid and strong acid. The ionic liquid can be modulated in order to tune the acidity, solubility, and interfacial properties, resulting in the enhanced results. The ionic liquid can be changed to tune the acidity and solubility such that the feed components are soluble in the catalyst mixture while the C₈ alkylate products are not. It has now been found that large amounts of either H₂SO₄ or triflic acid can be dissolved with ionic liquids to form homogeneous compositions and reacted with strong acids. This behavior allows fine tuning of the acidity and properties of the ionic liquid phase.

Further, the usage of the strong acid was shown to be capable of being reduced by nearly 25-35 wt. % when substituted with an ionic liquid. Since the use of strong acids is unfavorable for a variety of reasons, the use of ionic liquids to replace significant amounts of strong acid usage is desirable. The reduction in strong acid amount also improves safety by the ionic liquid reducing the vapor pressures of the acid mixture.

Another benefit is that the binary ionic liquid and strong acid catalyst mixture shows better catalyst recyclability, and can be used repeatedly. It was surprisingly and unexpectedly found that the ionic liquid had long-term stability over an extended period of recycle use. The catalyst recycling studies show good maintenance/reproducibility of conversion and selectivity for 25+ cycles for the Brønsted acid ionic liquid and strong Brønsted acid mixture compared to less than 5 cycles for the mineral acid alone. While the studies found that up to 25+ recycles maintained catalyst stability, it is conceived that many more recycles may be achieved with improved recycling. The stability of the catalyst can be increased by sometimes greater than 25+% with the ionic liquid/acid mixtures over the pure acid. The recycled catalyst mixture still provides stable olefin conversion (>95%), C₈ alkylates selectivity (>70%) and trimethylpentane/dimethylhexane selectivity (TMP/DMH>7) over a plurality of usage cycles. The recyclability of the ionic liquid and strong acid mixture is also environmentally friendly.

Ionic liquid catalyst systems have also been used for isoparaffin—olefin alkylation reactions. Patents that disclose a process for the alkylation of isoparaffins by olefins are U.S. Pat. No. 5,750,455 and U.S. Pat. No. 6,028,024, all of which are incorporated herein by reference. The present invention is an improvement in the synergistic benefit of combining a strong acid with an ionic liquid. In a more particular embodiment, the present invention can include a binary catalyst system having an ionic liquid and a strong Brønsted acid. In another particular embodiment, the present invention can include a binary catalyst system having a Brønsted acid ionic liquid and a strong Brønsted acid. The strong Brønsted acid is preferably a liquid acid.

A Brønsted acid is a molecule or ion that is able to lose, or “donate” a hydrogen cation (proton, H⁺). A wide range of compounds can be classified in the Brønsted-Lowry framework: mineral acids and derivatives, sulfonates, phosphonates, carboxylic acids, amines, carbon acids, 1,3-diketones, acetylacetone, ethyl acetoacetate, and many more.

A Brønsted base is a molecule or ion that is able to gain or “accept” a hydrogen cation (proton, H⁺).

Lewis acid is a molecule or ion that is an electron-pair acceptor. As used herein, a “Lewis acid” is a Lewis acid that is not also a Brønsted acid.

A Lewis base is a molecule or ion that is an electron-pair donor.

Brønsted acid ionic liquid is a Brønsted acid that is liquid at ambient conditions, such as sulfuric acid, HCl, HBr, HF, HI, phosphoric acid, or trifluoromethanesulfonic (triflic) acid

An example of an ionic liquid includes [HMIm][Tf₂H]. However, in the mixture with the strong acids, there may be some level of anion exchange. For instance with sulfuric acid: [HMIm][Tf2N]+H2SO4->[HMIm][HSO4]+HTf2N. HTf2N is a superacid itself. Other neutral ILs may not have potentially interesting exchanges.

It may be advantageous to include all the bronsted acid ionic liquids with different acidity and neutral ionic liquids. The roles of ILs in these reactions were tuning the acidity and the solubility of paraffin and olefin in the catalysts. So all the ILs may have the ability to play this role in the reactions.

Strong Brønsted acids include HCl, HBr, HF, HI, phosphoric acid, and trifluoromethanesulfonic (triflic) acid. fluoro-sulfonic acid (HSFO₃), 1,1,1-trifluoro-N-(trifluoromethylsulfonyl)methanesulfonamide (H(Tf2N)), or the like. In one aspect, the Brønsted acid is a superacid can have a Hammett number less than about negative ten (e.g., H₀<−10). The pKa of the acid can be less than that of benzoic acid (e.g., pKa about 4), where benzoic acid is excluded.

Ionic liquids are organic salts composed of relatively large organic cations and inorganic or organic anions that are liquid near room temperature (Tₘ<100° C.). They have virtually no vapor pressure and are molecularly tunable for desired properties. The combination of the cation and anion determines solubility, density, viscosity, and other properties of the liquids. There are several major classes of ionic liquid cations, and each contains the possibility to add any number of functional groups. Any change of either the structure or the length of the carbon side chain of both ions leads to different chemical or physical properties. Depending on the application, ionic liquids as designer solvents and catalysts can be tailored to meet the specifications.

The ionic liquids can be amine based, such as those formed by reacting a nitrogen-containing heterocyclic ring (cyclic amines), preferably nitrogen-containing aromatic rings (aromatic amines), with an alkylating agent (for example, an alkyl halide) to form a quaternary ammonium salt, followed by ion exchange with Bronsted Lewis acids and halide salts or anion metathesis reactions with the appropriate anion sources to form ionic liquids. The anion exchange is either with HA or MA (where H is hydrogen: Bronsted acid and M is any metal). Examples of suitable aromatic amines include pyridine and its derivatives, imidazole and its derivatives, and pyrrole and its derivatives. These amines can be alkylated with varying alkylating agents to incorporate a broad range of alkyl groups on the nitrogen including straight, branched or cyclic C₁₋₂₀ alkyl group, but preferably C₁₋₁₂ alkyl groups since alkyl groups larger than C₁-C₁₂ may produce undesirable solid products rather than ionic liquids. Pyridinium, oxazolium, thiaoxazolium, ammonium, phosphonium, and imidazolium-based ionic liquids are perhaps the most commonly used ionic liquids. Other amine-based ionic liquids including cyclic and non-cyclic quaternary ammonium and quaternary phosphonium salts are frequently used. Phosphonium and sulphonium-based ionic liquids have also been used.

In the present invention, the ionic liquids are Bronsted acids, either through the addition of an acidic group directly to the cation or anion and/or in combination with strong acids. Examples of acidic ionic liquids that can be used in the process of the present invention include, but are not limited to, [BMIm]HSO₄ (1-butyl-3-methylimidazolium hydrogen sulphate), [MBSIm]HSO₄ (1-methyl-3-(butyl-4-sulfonate) imidazolium hydrogen sulphate), [MBSIm]OTf (1-butyl-3-(butyl-3-sulfonyl)imidazolium trifluoromethanesulfonate), [MPSIm]OTf (1-butyl-3-(butyl-3-sulfonyl)imidazolium trifluoromethanesulfonate), and [OMIm] HSO₄ (1-octyl-3-methylimidazolium hydrogen sulphate).

The ionic liquid may improve various thermodynamic and interfacial properties of the acids such as: the dispersibility of paraffin or isoparaffin (e.g., isobutene) in the ionic liquid medium; more advantageous density differences between hydrocarbon phase and catalyst phase; reduced volatility of the acid; and improved miscibility between paraffin or isoparaffin (e.g., isobutene) and catalyst.

The properties of ionic liquids can be tailored by varying the cation and anion pairing. Ionic liquids and some of their commercial applications are described, for example, in J. Chem. Tech. Biotechnol, 68:351-356 (1997); J. Phys. Condensed Matter, 5:(supp 34B):B99-B106 (1993); Chemical and Engineering News, Mar. 30, 1998, 32-37; J. Mater. Chem., *:2627-2636 (1998); and Chem. Rev., 99:2071-2084 (1999), the contents of which are hereby incorporated by reference.

In one embodiment, isobutane/1-butene alkylation with ionic liquids and strong acids can be performed under the following scenarios: (1) the ionic liquids are acidic ionic liquids; (2) mixtures of acidic ionic liquids with strong acids such as H₂SO₄ or HCF₃SO₃. The conversion and selectivity obtained in of these cases is compared with those obtained with pure strong acids at similar pressure, temperature and feed composition. It has been found that surprisingly and unexpectedly the combination of acidic ionic liquids and strong acids provide synergistic improvement in yield and selectivity.

### Alkylation Process

In the alkylation process of the present invention, one or more feedstocks can include a reactive olefinic hydrocarbon. The olefinic groups provide the reactive sites for the oligomerization and alkylation reactions. The olefinic hydrocarbon can be a substantially pure olefinic hydrocarbon, or can be a mixture of hydrocarbons having different chain lengths and, thus, can have a wide boiling range. The olefinic hydrocarbon can be terminal olefin (an alpha olefin) or can be internal olefin (internal double bond). Exemplary olefins include, but are not limited to propylene, isobutylene, 1-butene, trans-2-butene, and cis-2-butene, pentenes, hexenes, heptenes, octenes, or the like. The olefinic hydrocarbon can be either straight chain or branched or a mixture of both.

One or more feedstocks can include a paraffin, such as an isoparaffin. The simplest isoparaffin is isobutane. Isopentanes, isohexanes, and other higher isoparaffins are also useable in the alkylation process. Mixtures of light isoparaffins can also be used in the present invention. Mixtures, such as C₄-C₅ isoparaffins, can be used.

The isoparaffins feed stream may also contain diluents such as normal paraffins. This can be a cost savings by reducing the cost of separating isoparaffins from close boiling paraffins, where the alkylate can more easily be separated from the paraffin. Normal paraffins will tend to be unreactive diluents in the process of the present invention.

Alkylation conditions for the process of the present invention include a temperature of from about −10° C. to about 200° C., preferably from about 10° C. to about 150° C., more preferably from about 20° C. to about 40° C., and most preferably from about 25° C. to about 35° C.: Alkylation conditions for the process of the present invention include a pressure of a range from about 1 to about 100 bar, about 5 bar to about 50 bar, or more preferably about 10 to about 30 bar.

Calculations of the conversions, selectivities, and TMP/DMH ratios are set forth in Clark and Subramaniam, Extended Alkylate Production Activity during Fixed-Bed Supercritical 1-Butene/isobtante Alkylation on Solid Acid Catalysis using Carbon Dioxide as a Diluent, Ind. Eng. Chem. Res. 1998, 37 1243-1250, which is incorporated by reference. The conversion is determined by determining the moles of 1-butene starting material, and then measuring the amount of 1-butene at the end of the reaction. Of the products, the Cg selectivity refers to the fraction of products that are C₈. Lastly, the TMP:DMH ratio refers to the mole ratio of these isomers in all of their forms.

The ionic liquids can be prepared by covalently tethering sulfonic acid groups to ionic liquid cations to produce Brønsted acidic ionic liquids. Such ionic liquids have high activities in esterification, alkylation of phenol with tert-butyl alcohol, and Friedel-Crafts alkylation of aromatic compounds with alkene.

In the studies described herein, different types of acidic ionic liquids synthesized, where the acidic group is found in either the cation or anion: [MPSIm][OTf], [MBSIm][OTf], [MBSIm][HSO₄] and [BMIm][HSO₄]. [MPSIm][OTf] and [MBSIm][OTf] have an acidic sulfonic acid group tethered by either a propyl or butyl group to the imidazolium cation. [BMIm][HSO₄] has acidity from the hydrogen sulfate group.

The alkylation using [BMIm][HSO₄] as the catalyst resulted in a low conversion, ˜11%, even after 20 minutes (compared with 10 minutes for the pure H₂SO₄) with a C₈ selectivity of only 8.7% and no trimethylpentanes produced. [MBSIm][OTf], with a butylsulfonic acid group attached to the cation and the triflate anion, also yielded low conversions and C₈ selectivity while [MPSIm][OTf] with a propylsulfonic acid group, yielded only slightly better conversion and selectivities. Thus, the conversions and selectivities with these acidic ILs alone are much lower than those obtained using just the strong acids. However, it should be noted that this is based upon an equal mass basis. When converted to a unit or concentration based upon acidic protons, the results become proportionally more similar.

When acidity was manifested in both of the cation and anion, as in the case of [MBSIm][HSO₄], the C₈ selectivity and TMP/DMH ratio were actually slightly better than that for pure sulfuric acid. However, the olefin conversion was still significantly lower.

When the alkylation is performed in the neutral ionic liquid, [HMIm][Tf₂N], without the addition of any catalyst, there was virtually no reaction (<0.5% olefin conversion). Increasing the fraction of H₂SO₄ provided higher conversion rates were obtained until approximately 50 wt % of the acid, after which little improvement is seen in the activity. Thus, less acid catalyst seems to suffice to achieve the same level of activity/conversion. The selectivity and TMP/DMH ratio significantly increase with increased fraction of the acid. This trend is possibly due to the preferential solubility of 1-butene over isobutane in the neutral ionic liquid relative to the pure acid. This neutral ionic liquid is known to have moderate solubility of olefins and lower solubility of alkanes. This equilibrium scenario would lead to isobutane starvation in the catalytic phase and encourage 1-butene dimerization. Accordingly, it is preferable that a neutral ionic liquid is excluded.

For ionic liquids with the sulfonic acid group, [MPSIm][OTf] and [MBSIm][OTf], higher proportions of sulfuric acid increase both the conversion and selectivities throughout the concentration range. At approximately 70 wt % of H₂SO₄ in the ionic liquid mixture, the conversion and selectivities are similar to those obtained with the pure acid. This suggests the possibility of a significant reduction in acid usage for commercial alkylations. Using the slightly acidic [HSO₄] anion in [BMIm][HSO₄] and mixing it with H₂SO₄, again increases the olefin conversion up to approximately 50 wt % acid. However, a maximum in the C₈ selectivity is seen at approximately 58 wt % H₂SO₄ with moderate TMP/DMH ratios. At ˜79 wt % H₂SO₄, the C₈ selectivity significantly drops, but yields higher TMP:DMH ratios.

The ionic liquid [MBSIm][HSO₄] have acidic groups in both the cation and anion. It intrinsically yields selectivities higher than pure sulfuric acid, but with lower olefin conversion. Higher proportions of H₂SO₄ in the ionic liquid increase the conversion, but initially reduce the selectivities. However, at approximately 60 wt % H₂SO₄ in the ionic liquid, the conversion seems to plateau, but the selectivities are at a minimum. The selectivities increase with further amounts of acid, and at ˜74 wt % acid, the conversion and selectivities match those of pure H₂SO₄. The foregoing results clearly demonstrate that the catalytic ionic liquid/acid mixtures can be highly tunable for conversion, C₈ selectivity, and TMP/DMH ratio by changing the structure and acidity of the cation and anion and by varying the proportion of acid.

Mixtures of the acidic ionic liquids with triflic acid can be used for the isobutane/1-butene alkylation using the cation, [MBSIm]. [MBSIm][OTf], when mixed with triflic acid, increased the conversion while maintaining consistent C₈ selectivity and only a slight drop in the TMP:DMH ratio. The selectivities obtained with roughly 67 wt % triflic acid are significantly better than those obtained with either the neat triflic acid or neat ionic liquid.

The long chain 1-octyl-3-methyl-imidazolium hydrogen sulfate ionic liquid ([OMIm][HSO₄]), which has a slightly acidic anion, when mixed with triflic acid at approximately 36 wt % and 76 wt % acid yielded conversion and selectivities that surpass those obtained with pure triflic acid. The acidic ionic liquids appear to mitigate the poorer selectivity found with triflic acid, by possibly adjusting the high acid value of the pure triflic acid. The ionic liquids may also be tuned to promote better solubility for the isobutane in the acid mixture.

The addition of ionic liquids to strong acids can be used to tune the acidity, reactant/product solubility, surface activity, density and viscosity. The low amounts of ionic liquid additive result in a dramatic improvement of product yield. The acidity of the catalyst can be fine-tuned by choice of the amount of ionic liquid additive and by choice of its anion. In a similar manner, it can be possible to tune the solubilities of the alkylation reactants and products by a suitable choice of the ions of the ionic liquid and by the amount of added ionic liquid.

Among the ionic liquids used for creating the binary mixtures with conventional acids, the [OMIm][HSO₄] showed the best combination of activity and C₈ alkylate selectivity. The improvement may be from improved isobutane solubility in the binary mixture compared to other ionic liquid/acid mixtures. For isobutene+2-butene alkylation, ionic liquids bearing a larger alkyl group on their cation ([OMIm]) may have relatively higher activity than a smaller one ([HMIm] or [BMIm]) with the same anionic composition. This may be due to the higher solubility of reactants, especially isobutane, in the [OMIm]-based ionic liquid. In general, the solubility of hydrocarbons increases significantly with increasing length of the alkyl group in the ionic liquids. For a series of 1-alkyl-3-methylimidazolium cations, increasing the alkyl chain length from butyl to hexyl to octyl increases the hydrophobicity.

Also, higher I/O ratios favor improved isooctane yields.

The ability of the ionic liquid/acid mixtures to be recycled and still be efficient for the conversion, selectivity, and TMP/DMH ratio of the alkylate is a surprising and unexpected benefit. [OMIm][HSO₄] and H₂SO₄ can be used at a feed I/O ratio of about 7.5. The isobutane/1-butene mixtures can be reacted and the reactant/product phase, decanted and removed. Fresh reactants can be added to start the next run.

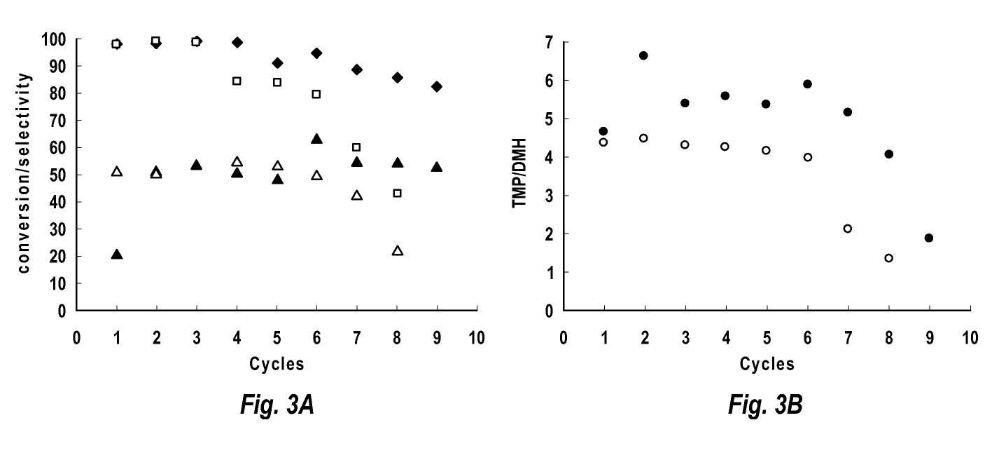

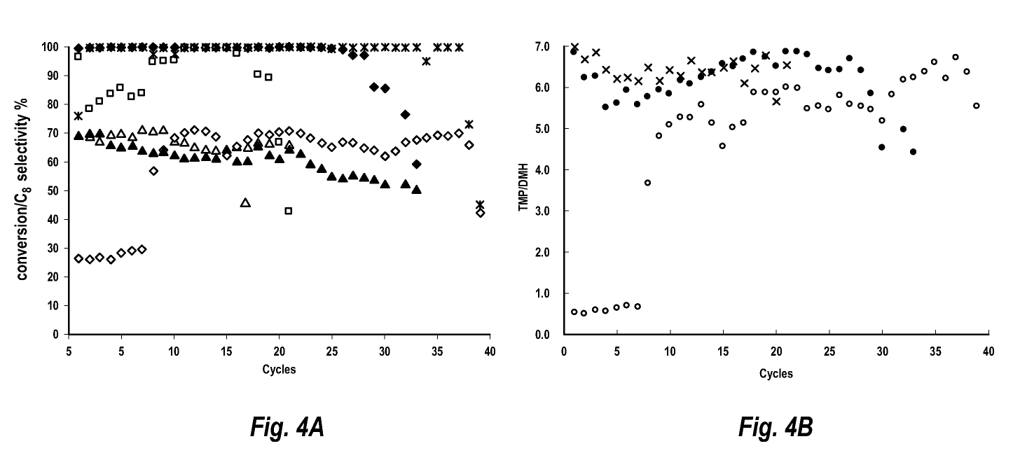

FIGS. 3A-3B illustrate the results of recycling the ionic liquid/acid mixture up to nine times as compared with the results of just the pure sulfuric acid. Similar conversion and selectivity were observed for both the ionic liquid/acid mixture and the pure acid for about four recycles, although the TMP/DMH ratio is better in the ionic liquid/acid mixture. However, after four recycle runs, the pure sulfuric acid conversion and selectivities significantly decreased. After eight recycles, the conversion and selectivity for pure sulfuric acid were about half of their values observed during the first run. However, for the ionic liquid/acid mixture, the conversion only decreased by approximately 20% by recycle 9, with nearly unchanged C₈ selectivity, although with reduced TMP/DMH ratio. It is thought that tuning can result in maintained conversion and TMP/DMH ratio.

[OMIm][HSO₄] was also used in mixtures of triflic acid with a feed I/O ratio of 7.5. FIGS. 4A-4B illustrate that even after 25 recycles, the ionic liquid/acid mixture maintains high conversion and selectivity with better TMP:DMH selectivity than the pure triflic acid. This mixture also does not suffer from the initial low conversion and selectivity that affects the pure triflic acid. The high initial acidity of the pure triflic acid leads to cracking reactions which degrades the selectivity, but the ionic liquid seems to moderate this. The spent [OMIm][HSO₄]/CF₃SO₃H catalyst was slightly colored after all of the recycling runs. However, the spent sulfuric acid was opaque. This suggests that the deactivation of the mixed ionic liquid/triflic acid catalyst may be partly due to the entrainment of heavy compounds in the acidic solution. In contrast, the lifetime of the H₂SO₄ was only 3-6 cycles (FIGS. 3A-3B). Further, the C₈ selectivity and the TMP/DMH ratio obtained with the binary [OMIm][HSO₄]/CF₃SO₃H mixture were higher than those obtained with H₂SO₄. In addition, the [OMIm][HSO₄]/triflic acid system demonstrated a stable conversion and C₈ selectivity as shown in FIGS. 4a-4B.

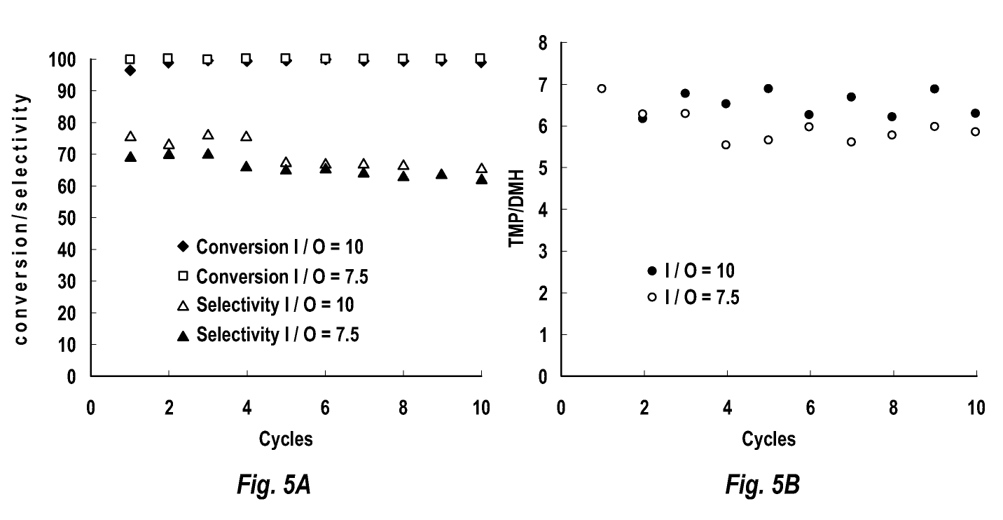

FIGS. 5A-5B illustrate that the conversion and selectivities obtained at the higher I/O ratio of I/O were nearly identical to that the values obtained at an I/O ratio of 7.5. The ability to use lower I/O ratios without sacrificing alkylate yields and stability is desirable to lower isobutane separation costs.

### EXAMPLES

Six different types of ionic liquids were investigated representing 5 different cations and 3 different anions as shown in FIG. 1. [BMIm][HSO₄] (1-butyl-3-methylimidazolium hydrogen sulphate) was purchased commercially from Sigma-Aldrich Co. The others were synthesized.

### 1-n-Alkyl-3-Methyl-Imidazolium Halide [R-MIm][X]

Ionic liquids used in this study were prepared by anion exchange from the corresponding bromide or chloride salts of the imidazolium cations ([R-MIm][X]) with different n-alkyl substituents as known. The bromide or chloride salt of the imidazolium cation was prepared from a quaternization reaction of 1-methylimidazole with a slight excess of the corresponding alkyl halide in acetonitrile at 313.15 K under argon atmosphere with stirring for three days. The solvent was removed in a rotary evaporator under reduced pressure at 40° C. which was further evacuated by connection to a high vacuum (<10⁻⁴ Torr) at 50° C. for at least 48 hours.

### 1-Hexyl-3-Methylimidazolium Bis(Trifluoromethylsulfonyl)ImideAmide ([HMIm] [Tf₂N])

[HMIm][Tf₂N] was prepared from the anion exchange of [HMIm][Br] with Li[Tf₂N] in deionized water as knows. The denser hydrophobic ionic liquid phase is decanted and washed six to eight times with approximately twice the volume of water compared to the ionic liquid. The ionic liquid is then dried under vacuum. ¹H NMR chemical shifts (relative to TMS internal standard) and coupling constants J/Hz: δ=8.65 (s, 1H), 7.39 (d, 2H, J=4.19), 4.17 (q, 2H, J=7.4), 3.93 (s, 3H), 1.87 (m, 4H), 1.32 (m, 6H) 0.87 (t, 3H, J=6.53). Analysis calculated for C12H19N3F6S2O4: C, 32.2; H, 4.28; N, 9.39; S, 14.33. Found: C, 32.21; H, 4.27; N, 9.25; S, 14.19; water content is less than 100 ppm, and Br content is less than 20 ppm.

### 1-Butyl-3-Methylimidazolium hydrogensulfate ([BMIm][HSO4])

[BMIm][HSO₄] was prepared from the anion exchange of [BMIm][Cl] with H₂SO₄ in solvent. The ionic liquid is then dried under vacuum. ¹H NMR chemical shifts (relative to TMS internal standard) and coupling constants J/Hz: S=8.65 (s, 1H), 7.39 (d, 2H, J=4.19), 4.17 (q, 2H, J=7.4), 3.93 (s, 3H), 1.87 (m, 4H), 1.32 (m, 6H) 0.87 (t, 3H, J=6.53); water content is less than 100 ppm.

### 1-Octyl-3-Methylimidazolium hydrogensulfate ([OMIm][HSO₄])

[OMIm][HSO₄] was prepared from the anion exchange of [OMIm][Cl] with H₂SO₄ in deionized water. The ionic liquid is then dried under vacuum. ¹H NMR chemical shifts (relative to TMS internal standard) and coupling constants J/Hz: δ=8.65 (s, 1H), 7.39 (d, 2H, J=4.19), 4.17 (q, 2H, J=7.4), 3.93 (s, 3H), 1.87 (m, 4H), 1.32 (m, 6H) 0.87 (t, 3H, J=6.53); water content is less than 100 ppm.

### 3/4-(3-methyl-Imidazoliumyl)propyl (or butyl)-1-sulfonate

Brønsted acidic imidazolium ionic liquids were prepared by attaching alkyl-sulfonic acid to the imidazolium ring through a zwitterionic intermediate. 1,3-Propane sultone or 1,4-butane sultone was combined with 1-methylimidazole in equimolar quantities in toluene forming the zwitterionic imidazolium sulfonate which precipitates upon formation. After the reaction, the solid was washed three times with diethyl ether and toluene to remove any unreacted starting materials, and the solid was dried in vacuo.

### 3-Methyl-1-(3-Sulfobutyl)-imidazolium hydrogen sulfate ([MBSIm][HSO₄])

[MBSIm][HSO₄] was prepared as known. The zwitterionic intermediate as described above was added in a stoichiometric ratio to H₂SO₄ in solvent. The ionic liquid is then dried in vacuo. ¹H NMR chemical shifts (relative to TMS internal standard) and coupling constants J/Hz: δ=8.65 (s, 1H), 7.39 (d, 2H, J=4.19), 4.17 (q, 2H, J=7.4), 3.93 (s, 3H), 10.87 (m, 4H), 1.32 (m, 6H) 0.87 (t, 3H, J=6.53); water content is less than 100 ppm.

### 3-Methyl-1-(3-Sulfobutyl)-imidazolium trifluoro methylsulfonate([MBSIm][OTf])

[MBSIm][OTf] was prepared in a known method. The zwitterionic intermediate as described above was added in a stoichiometric ratio to an pure trifluoromethylsulfonic acid (triflic acid) in solvent at 40° C. The ionic liquid phase was then washed repeatedly with toluene and ether to remove non-ionic residues, and dried in vacuo. NMR chemical shifts (relative to TMS internal standard) and coupling constants J/Hz: δ=8.65 (s, 1H), 7.39 (d, 2H, J=4.19), 4.17 (q, 2H, J=7.4), 3.93 (s, 3H), 1.87 (m, 4H), 1.32 (m, 6H) 0.87 (t, 3H, J=6.53); water content is less than 100 ppm.

### 3-Methyl-1-(3-Sulfopropyl)-imidazolium trifluoromethylsulfonate([MPSIm][OTf])

[MPSIm][OTf] was prepared in a known method. The zwitterionic intermediate as described above was added in a stoichiometric ratio to an pure trifluoromethylsulfonic acid (triflic acid) in solvent at 40° C. The ionic liquid phase was then washed repeatedly with toluene and ether to remove non-ionic residues, and dried in vacuo. ¹H NMR chemical shifts (relative to TMS internal standard) and coupling constants J/Hz: δ=8.65 (s, 1H), 7.39 (d, 2H, J=4.19), 4.17 (q, 2H, J=7.4), 3.93 (s, 3H), 1.87 (m, 4H), 1.32 (m, 6H) 0.87 (t, 3H, J=6.53); water content is less than 100 ppm.

### Alkylation Apparatus and Procedure

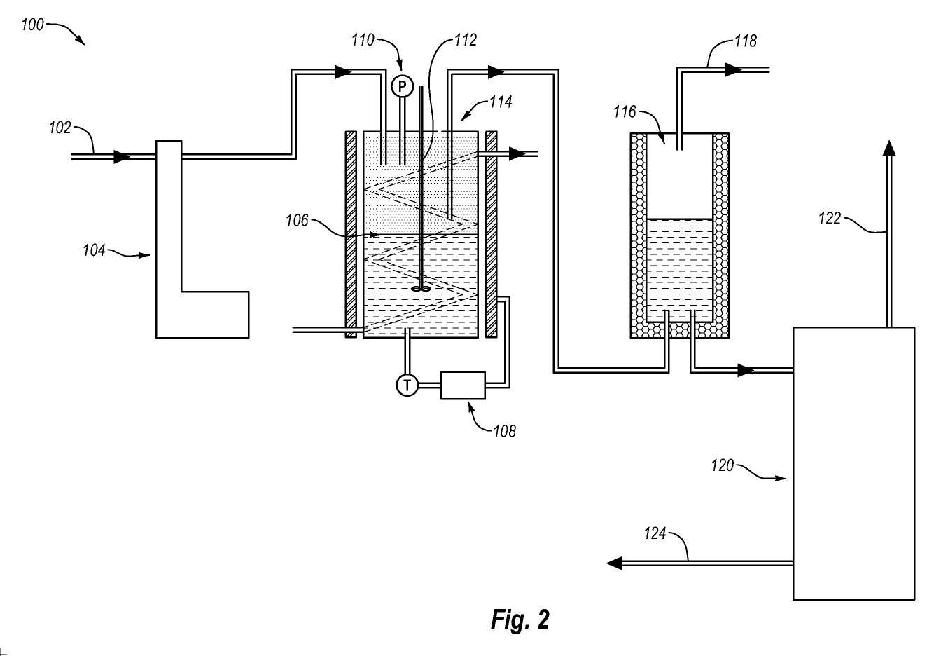

Isobutane/1-butene alkylations were performed in a 50 mL stirred autoclave reactor as shown in FIG. 2. Temperature and pressure were controlled and monitored with a Camile 2500 data acquisition system. Cooling was provided by a re-circulating chiller using ethylene glycol or propylene glycol. Due to the exothermicity of the reaction, there was a 2-4° C. increase in temperature upon reaction. The isobutane and 1 butene feeds (99+%) were premixed in a storage vessel to a specified I/O ratio, analyzed offline, and then transferred to a syringe pump and cooled to −5° C. The reactor effluent was collected in a trap chilled by a dry-ice+acetone bath. The products were analyzed offline by gas chromatography (GC) with a Varian CP-3380 GC, equipped with a flame ionization detector, and a DB-Petro 100 m column (J&W Scientific). Helium is used as the GC carrier gas and as the flame ionization detector (FID) makeup gas. The analysis conditions were: split ratio=50:1, injector temperature=250° C., detector temperature=300° C., carrier gas flow rate=20 sccm. The temperature program for GC analysis is as follows: initial column temperature 30° C./hold for 15 min, 0.5° C./min to 100° C., then 5° C./min to 300° C./hold for 15 min. An alkylate reference standard (Supelco) allowed identification of the trimethylpentanes (TMP) and dimethylhexanes (DMH). The GC area percent was equated to weight percent since all hydrocarbons in the reactor effluent have response factors close to unity. The combined mass of TMP and DMH is referred to as the “alkylate product”. Propane, an impurity in the isobutane feed, was used as an internal standard for butene conversion calculations. The butene isomers were lumped together when calculating butene conversion.

All experiments were performed in batch mode, however, the process could be performed in a semi-continuous or continuous process. A typical experiment begins with the addition of the acid, ionic liquid, or acid/ionic liquid mixture. The reactor was sealed and cooled to a desired temperature. Approximately 20 mL of the premixed hydrocarbon feed at a desired isobutane/1-butene ratio was pumped into the reactor at a flow rate of 5 mL/min with stirring of the liquid phase. After a certain reaction time (10-30 min), stirring was stopped and the mixture allowed to settle for 5 min. to let the catalyst separate from the hydrocarbons. The hydrocarbon layer was then led to an external trap cooled with dry ice/acetone solution. Care was taken to avoid outflow of the catalyst layer. For recycling studies, the vessel was filled with fresh feed and the procedure repeated.

### Chemical Analysis

H-NMR spectra were recorded on a Bruker 400 NMR Spectrometer using TMS as a reference for H chemical shifts. The water content was determined by a Mettler Toledo DL32 Karl Fisher Coulometer and the Br content was measured by a Cole Parmer Bromide Electrode (27502-05) equipped with an Oakton Ion 510 series meter.

To provide benchmarks for the novel ionic liquid acid systems, the alkylation of isobutene with 1-butene was performed with traditional catalysts: sulfuric acid and triflic acid. The results are shown in Entries 1 and 2 of Table 1 below (Table 1 Common conditions: Initial Volume of Hydrocarbon Feed=20 mL; I/O in feed=10; P=2 bar; Initial T=8° C.).

At 8° C. and an I/O ratio of 10, nearly quantitative conversion is achieved in just over 10 minutes for both catalysts. However, the selectivity for each is markedly different. Sulfuric acid provided a selectivity of nearly 60% to octanes with a trimethylpentanes (TMP)/dimethylhexanes (DMH) ratio of approximately 6. These results are consistent with the alkylate quality of a typical commercial plant. However, the C₈ selectivity and the TMP/DMH ratio obtained with triflic acid were approximately 41% and 1.4, respectively. These values are similar to those previously obtainable. Selectivity and alkylate quality are strong functions of acid strength. The highest C₈ alkylates selectivity can occur at an acid strength, given by the Hammett acidity scale, of H₀=−10.7. At higher acidity however, cracking reactions are favored and hence lighter components are also observed in the product stream. Triflic acid is a strong acid with H₀=−14.1. Consequently, the C₈ alkylates selectivity obtained with pure triflic acid is lower.

It will be understood, by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

This research was partly supported by the National Science Foundation Engineering Research Center Grant (EEC-0310689).

### TABLES

TABLE 1
Isobutane/1-butene alkylation results

| | | | | | | Batch | 1-Butene | C₈ | |
|---|---|---|---|---|---|---|---|---|---|
| | Total Catalyst | Ionic Liquid | Wt % | | Wt % | Time | Conversion | Selectivity | TMP/DMH |
| Entry | Weight (g) | (IL) | IL | Acid | Acid | (min) | (%) | (%) | Selectivity |
| 1 | 27.01 | None | | H₂SO4 | 100.0 | 10 | 97.3 | 61.7 | 6.1 |
| 2 | 24.87 | None | | CF₃SO₃H | 100.0 | 10 | 98.7 | 41.1 | 1.4 |
| 3 | 25.84 | [HMIm][Tf2N] | 100.0 | None | 0.0 | 30 | 0.3 | 15.4 | 0.5 |
| 4 | 27.61 | [HMIm][Tf2N] | 93.6 | H₂SO4 | 6.4 | 10 | 2.2 | 15.9 | 0.6 |
| 5 | 45.43 | [HMIm][Tf2N] | 60.8 | H₂SO4 | 39.2 | 30 | 59.0 | 19.2 | 5.8 |
| 6 | 30.75 | [HMIm][Tf2N] | 42.0 | H₂SO4 | 58.0 | 20 | 89.4 | 31.1 | 4.8 |
| 7 | 39.01 | [HMIm][Tf2N] | 8.7 | H₂SO4 | 91.3 | 20 | 89.7 | 60.2 | 5.1 |
| 8 | 31.05 | [MPSIm]OTf | 100.0 | None | 0.0 | 20 | 6.3 | 11.0 | 0.8 |
| 9 | 31.84 | [MPSIm]OTf | 43.6 | H₂SO4 | 56.4 | 20 | 93.7 | 43.6 | 3.6 |
| 10 | 36.43 | [MPSIm]OTf | 26.8 | H₂SO4 | 73.2 | 20 | 92.2 | 63.9 | 5.1 |
| 11 | 28.86 | [MPSIm]OTf | 100.0 | None | 0.0 | 20 | 2.2 | 6.6 | 0.0 |
| 12 | 30.22 | [MPSIm]OTf | 70.4 | H₂SO4 | 29.6 | 20 | 8.2 | 31.2 | 2.7 |
| 13 | 32.86 | [MPSIm]OTf | 46.7 | H₂SO4 | 53.3 | 20 | 91.3 | 41.0 | 3.8 |
| 14 | 37.39 | [MPSIm]OTf | 28.1 | H₂SO4 | 71.8 | 20 | 91.1 | 57.0 | 4.8 |
| 15 | 24.47 | [BMIm]HSO4 | 100.0 | None | 0.0 | 20 | 11.4 | 8.7 | 0.1 |
| 16 | 27.61 | [BMIm]HSO4 | 67.6 | H₂SO4 | 32.4 | 20 | 51.8 | 22.4 | 5.2 |
| 17 | 30.93 | [BMIm]HSO4 | 41.7 | H₂SO4 | 58.3 | 20 | 91.0 | 60.3 | 4.9 |
| 18 | 33.29 | [BMIm]HSO4 | 20.9 | H₂SO4 | 79.1 | 20 | 90.4 | 41.0 | 7.9 |
| 19 | 27.1 | [MBSIm]HSO4 | 100.0 | None | 0.0 | 20 | 5.4 | 62.0 | 7.2 |
| 20 | 25.49 | [MBSIm]HSO4 | 63.9 | H₂SO4 | 36.1 | 20 | 12.1 | 27.2 | 8.1 |
| 21 | 30.26 | [MBSIm]HSO4 | 40.9 | H₂SO4 | 59.2 | 20 | 89.5 | 25.9 | 2.6 |
| 22 | 36.14 | [MBSIm]HSO4 | 26.0 | H₂SO4 | 74.0 | 20 | 95.6 | 62.3 | 6.4 |
| 23 | 26.85 | [MBSIm]OTf | 96.6 | CF₃SO₃H | 3.4 | 20 | 1.1 | 59.5 | 8.8 |
| 24 | 26.45 | [MBSIm]OTf | 81.3 | CF₃SO₃H | 18.7 | 10 | 68.3 | 57.9 | 6.2 |
| 25 | 29.11 | [MBSIm]OTf | 48.3 | CF₃SO₃H | 51.7 | 10 | 95.2 | 64.7 | 5.1 |
| 26 | 31.79 | [MBSIm]OTf | 33.3 | CF₃SO₃H | 66.7 | 10 | 93.3 | 72.1 | 5.7 |
| 27 | 29.32 | [BMIm]HSO4 | 23.2 | CF₃SO₃H | 76.8 | 10 | 98.1 | 72.4 | 8.1 |
| 28 | 20.58 | [OMIm]HSO4 | 67.6 | CF₃SO₃H | 32.4 | 10 | 85.1 | 61.9 | 6.2 |
| 29 | 24.51 | [OMIm]HSO4 | 59.6 | CF₃SO₃H | 40.4 | 10 | 91.2 | 65.6 | 6.8 |
| 30 | 25.07 | [OMIm]HSO4 | 23.7 | CF₃SO₃H | 76.3 | 10 | 96.3 | 75.8 | 6.8 |

1. A alkylation catalyst composition comprising:
an acid ionic liquid and
a strong Brønsted acid that is not an ionic liquid.

2. The catalyst composition of claim 1, wherein the acid ionic liquid is a Brønsted acid ionic liquid is selected from the group consisting of [BMIm]HSO₄ (1-butyl-3-methylimidazolium hydrogen sulphate), [MBSIm] HSO₄ (1-methyl-3-(butyl-4-sulfonate) imidazolium hydrogen sulphate), [MBSIm]OTf (1-butyl-3-(butyl-3-sulfonyl)imidazolium trifluoromethanesulfonate), [MPSIm]OTf (1-butyl-3-(butyl-3-sulfonyl) imidazolium trifluoromethanesulfonate), and [OMIm]HSO₄ (1-octyl-3-methylimidazolium hydrogen sulphate).

3. The catalyst composition of claim 1, wherein the acid ionic liquid is a protic ionic liquid.

4. The catalyst composition of claim 3, wherein the protic ionic liquid includes a [Cation][Acidic Anion] selected from the group consisting of [Cation][HSO₄], [Cation][OTf], [Cation][Tf₂N], [Cation][H₂PO₄], [Cation][HPO₄]⁻², [Cation][HCO₃], [Cation][Acidic Inorganic Anion], [Cation][H₂VO₄]

5. The catalyst composition of claim 1, wherein the strong Brønsted acid is selected from the group consisting of sulfuric acid, HCl, HBr, HF, HI, phosphoric acid, trifluoromethanesulfonic (triflic) acid fluoro-sulfonic acid (HSFO₃), 1,1,1-trifluoro-N-(trifluoromethylsulfonyl)methanesulfonamide (H(Tf2N)), or the like.

6. The catalyst composition as in claim 1, wherein the strong Brønsted acid has a Hammett number less than about negative ten (e.g., H₀<−10).

7. The catalyst composition of claim 1, wherein the strong Brønsted acid is present at more than about 50 wt % of the composition.

8. The catalyst composition of claim 1, wherein the alkylation catalyst composition is substantially devoid of a weak or medium Brønsted acid and/or substantially devoid of a neutral ionic liquid, and/or substantially devoid of a Lewis acid ionic liquid.

9. An alkylation reaction mixture comprising:
a. the catalyst composition of claim 1; and
b. an isoparaffin and an olefin.

10. The reaction mixture of claim 9, wherein the olefin is selected from the group consisting of propylene, pentene, isobutylene, 1-butene, trans-2-butene, cis-2-butene, and derivatives thereof.

11. The reaction mixture of claim 9, wherein the isoparaffin is selected from the group consisting of isobutane and isopentane

12. The reaction mixture of claim 9, wherein isoparaffin:olefin (“I:O”) ratio is 1:1, 2:1, 3:1, 4:1, 5:1, 6:1, 7:1, 8:1, 9:1, 10:1, 11:1, 12:1, 13:1, 14:1, 15:1, vice versa, or any range between a combination of the foregoing.

13. A process for alkylating an isoparaffin comprising:
providing a reaction mixture of claim 9;
reacting the isoparaffin and an olefin in the presence of the alkylation catalyst;
forming an alkylated isoparaffin product; and
recovering an alkylated isoparaffin from the reaction mixture.

14. The process of claim 13, wherein the olefin is selected from the group consisting of propylene, pentene, isobutylene, 1-butene, trans-2-butene, cis-2-butene, and derivatives thereof.

15. The process of claim 13, wherein the isoparaffin is selected from the group consisting of isobutane and isopentane.

16. The process of claim 13, wherein the reaction mixture has an isoparaffin/olefin (“I/O”) ratio of 1:1, 2:1, 3:1, 4:1, 5:1, 6:1, 7:1, 8:1, 9:1, 10:1, 11:1, 12:1, 13:1, 14:1, 15:1, vice versa, or any range between a combination of the foregoing.

17. The process of claim 13, wherein the reacting step occurs at a temperature of about −10° C. to about 50° C.

18. The process of claim 13, wherein the reacting step occurs at a pressure of about 1 bar to about 60 bar.

19. The process of claim 13, further comprising the step of recovering the alkylation catalyst from the reaction mixture.

20. The process of claim 13, further comprising recycling the recovered alkylation catalyst into a second reaction mixture with fresh isoparaffin and olefin.

21. The process of claim 20, further comprising recycling the recovered alkylation catalyst at least 5 times, and the reaction has a conversion of at least 50% in each cycle.

22. The process of claim 13, wherein the reacting step is conducted in a batch, semi-continuous, or continuous manner.

23. The process of claim 13, wherein the process has a C₈ selectivity which is greater than 50%, and C₈ selectivity is defined as the weight of C₈ products divided by the weight of all products.

24. The process of claim 13, wherein the conversion of the limiting reactant is greater than about 90%.

25. The process of claim 13, wherein the process has a trimethylpentane:dimethylhexane (“TMP:DMH”) ratio of greater than 4:1.
